(19) United States
(12) Patent Application Publication
(10) Pub. No.: US 2021/0316030 A1
(43) Pub. Date: Oct. 14, 2021

(54) PROTECTIVE BARRIER FOR STERILIZATION CONTAINERS

(71) Applicant: Billy W. WILLIAMS, Augusta, GA (US)

(72) Inventor: Billy W. WILLIAMS, Augusta, GA (US)

(21) Appl. No.: 17/269,883

(22) PCT Filed: Aug. 19, 2019
(86) PCT No.: PCT/US19/47018

§ 371 Date: Feb. 19, 2021

Related U.S. Application Data
(60) Provisional application No. 62/720,026, filed on Aug. 20, 2018.

(51) Int. Cl. A61L 2/28; A61L 2/07; A61L 2/14; A61L 2/20; B32B 1/00; B32B 5/02; B32B 5/26

(52) U.S. Cl. CPC A61L 2/28; B32B 2250/02; A61L 2/14; A61L 2/208; A61L 2/206; A61L 2/202; B32B 1/00; B32B 5/022; B32B 5/26; A61L 2202/24; A61L 2202/122; A61L 2202/14; B32B 2307/724; B32B 2307/7265; B32B 2439/40; B32B 2307/73; B32B 2262/0253; B32B 2307/54; A61L 2/07

(57) ABSTRACT

The present disclosure provides a cover or sleeve for use with a sterilization container comprising a collapsible enclosure configured to conform to the shape and size of the sterilization container. The collapsible enclosure includes an inner layer configured to withstand a sterilization procedure and a protective layer bonded to the inner layer. The protective layer is configured to protect the inner layer and to withstand the sterilization procedure. The enclosure further comprises a scalable opening for receiving the sterilization container within the interior of the enclosure. The collapsible enclosure is universally adapted for use for a variety of validated sterilization methods, such as steam, hydrogen peroxide, gas plasma, ozone, ethylene oxide and the like and with various sterilization trays, baskets and the like.

### TECHNICAL FIELD

The present invention relates to components for use with medical and/or dental sterilization systems, and more particularly, to an adaptable, sealable outer sleeve for enclosing and protecting a variety of different sterilization containers, such as instrument trays, baskets and the like.

### BACKGROUND OF THE INVENTION

Sterilization trays are commonly used to sterilize and store medical and/or dental instruments or implants. A sterilization tray system usually includes two main components: a container such as a tray, basket or base, and an outer cover for the container. These two components comprise materials suitable for steam sterilization or other known validated sterilization methods, such as ethylene oxide, gas plasma, ozone, hydrogen peroxide and the like. In typical sterilization tray systems, the tray and cover have vertical or near vertical walls on all sides that allow for the tray and cover to mate, forming an enclosed container. In addition, the tray and cover are usually perforated to allow high temperature gases and fluids to enter and exit the container. In order to withstand the high temperatures used in, for example, steam-based sterilization techniques, the tray and cover are typically made of a sheet metal or rigid thermoplastic material.

Unfortunately, sterilization tray systems are costly to manufacture and maintain. For example, conventional metal sterilization tray and lid designs are cumbersome and usually require a hinge or clip to couple them together. These mechanisms often break or fail after repeated cycles of sterilization, which exposes the internal contents of the tray to harsh temperatures and/or chemicals.

Furthermore, to accommodate various types of instruments and/or implants, the containers or enclosures may come in different heights, widths and depths depending on the type and size of instruments/implants to be sterilized, autoclaved, or lyophilized. The tray and cover may comprise separate components or they may be removably coupled to each other with one or more latching devices to secure the cover to the tray. Typically, the latches form handles to allow the tray system to be handled as a single unit. In addition, most tray systems are stackable, to accommodate storage and ease of use. This consequently requires medical facilities to carry a wide variety of different forms and shapes of trays and enclosures. Maintaining the matching components for the sterilization systems can be a costly and burdensome requirement for these facilities.

Another drawback with existing sterilization containers is that they typically need to be dried, cooled down and/or equalized after a sterilization procedure before they can be used again for the next procedure. The dry cycle, cool down and equalization times typically require almost 2-3 hours, which is wasted “down-time” wherein the containers are not available for sterilizing instruments. Thus, the facility may not have sterile instruments available for emergent or unplanned surgical procedures.

Accordingly, it would be desirable to provide an improved sterilization component or cover that can be universally adapted for use with conventional sterilization systems and containers such as trays, baskets and the like. It would be even more desirable to provide an outer sterilization cover that is less prone to mechanical failure and/or can be used to help reduce the “down-time” associated with conventional sterilization tray containers.

### SUMMARY OF THE INVENTION

The present disclosure provides a cover or sleeve for use with a sterilization container comprising a collapsible enclosure configured to conform to the shape and size of the sterilization container. The collapsible enclosure includes an inner layer configured to withstand a sterilization procedure and a protective layer bonded to the inner layer. The protective layer comprises a material configured to protect the inner layer and to withstand the sterilization procedure. The enclosure further comprises a sealable opening for receiving the sterilization container within the interior of the enclosure. The collapsible enclosure is universally adapted for use with existing sterilization containers designed to undergo validated sterilization methods, such as steam, hydrogen peroxide, gas plasma, ozone, ethylene oxide and the like. This allows medical facilities to sterilize instruments in a variety of different containers and using different sterilization methods, while minimizing or completely eliminating the down-time typically associated with dry cycles, cool-down and/or equalization periods.

The inner and protective layers preferably comprise materials that are substantially impermeable to liquids, pathogens and other particulates, while being permeable to the gases typically used for sterilization, such as steam, ethylene oxide or the like. This ensures that the sterilization container and the contents therein remain substantially dry throughout the procedure and sterile before use. In preferred embodiments, the inner and protective layers comprise materials configured to withstand temperatures of at least 270 degrees Fahrenheit for a minimum of 60 consecutive minutes without being compromised.

In a preferred embodiment, the inner layer comprises an anti-microbial hydrophobic material to protect the sterilization container from outside moisture and pathogens. The inner layer preferably includes anti-wicking properties sufficient to form a fluid and particulate barrier. In an exemplary embodiment, the inner layer comprises a non-woven polypropylene material.

The protective layer preferably comprises first and second layers bonded to opposite sides of the inner layer. The protective layers are each preferably made of a non-absorbent material with high tensile strength to withstand the rugged environment found in medical facilities, such as canvas or a similar material. In an exemplary embodiment, the protective layers are bonded to the inner layer with a medically-safe thermo bonding technique to secure the protective layers to the inner layer without affecting the integrity and functionality of the overall enclosure.

The sealable opening is movable between an open position, wherein the sterilization container may be inserted into the enclosure, and a closed position, wherein the opening is sealed to the outside environment and substantially impermeable to liquids, pathogens and other particulates. In one embodiment, the sealable opening comprises a self-adhesive strip configured to adhere to the protective layer. In another embodiment, the sealable opening comprises a clamp mechanism that includes a tamper resistant lock for locking the opening in the closed position until ready for use.

In certain embodiments, the collapsible enclosure may further include a sensor configured to detect pathogens within the interior of the enclosure when the sealed opening is in the closed position. The enclosure may further include an indicator coupled to the sensor and configured to indicate and display the presence of pathogens detected by the sensor. This allows the user to verify that the enclosure and sealable opening have remained secure from the time the opening was sealed to the time the enclosed items are ready for use in a procedure. Thus, once the sealable opening is clamped or otherwise sealed in the closed position, the collapsible enclosure will maintain sterility until it is reopened (i.e., sterility maintenance).

In another aspect of the invention, a sterilization container system comprises a sterilization container and a flexible outer sleeve configured to conform to an outer surface of the sterilization container. The outer sleeve comprises at least one layer configured to withstand a sterilization procedure and a sealable opening for receiving the sterilization container. The sterilization container may comprise a metal, plastic or similar material and may be a tray, basket or similar container. The flexible outer sleeve is configured to conform to a variety of different sterilization containers and to withstand validated sterilization methods, such as steam, hydrogen peroxide, gas plasma, ozone, ethylene oxide and the like.

The recitation herein of desirable objects which are met by various embodiments of the present invention is not meant to imply or suggest that any or all of these objects are present as essential features, either individually or collectively, in the most general embodiment of the present invention or in any of its more specific embodiments.

### BRIEF DESCRIPTION OF THE DRAWINGS

FIGS. 1A and 1B illustrate a sterilization cover for use with a sterilization container according to the present disclosure;

FIG. 2 is a side view of the sterilization cover of FIG. 1;

FIG. 3 is a bottom view of the sterilization cover of FIG. 1;

FIG. 4 is a perspective view of a clamp for use with the sterilization cover of FIG. 1 in accordance with one embodiment of the present invention; and

FIG. 5 illustrates a self-adhesive strip for use with the sterilization cover of FIG. 1 in accordance with another embodiment of the present invention.

### DETAILED DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Except as otherwise noted, any quantitative values are approximate whether the word “about” or “approximately” or the like are stated or not. The materials, methods, and examples described herein are illustrative only and not intended to be limiting. Any molecular weight or molecular mass values are approximate and are provided only for description.

The following disclosure is presented with respect to applications for surgical and/or dental instruments or implants. However, the embodiments may be implemented in any application requiring treatment of devices or items, especially for certain heat, steam, radiation, or chemical treatments.

### Introduction to the Sterilization System Embodiments

In general, the embodiments relate to an enclosure component for use with sterilization systems that typically include some form of a container, such as a tray or basket. The component may comprise a flexible outer sleeve or a collapsible enclosure that is universally adapted for use with existing sterilization containers of known medical and/or dental sterilization systems. The embodiments of the enclosure may be designed with materials and components that can withstand any form of sterilization technique. For example, the embodiments may be capable of sterilization using (1) ethylene oxide (EO); (2) chemical soaking; (3) steam sterilization; and (4) plasma sterilization, e.g., employing a hydrogen peroxide (H₂O₂) vapor.

In some embodiments, the enclosure is designed with materials and components for steam-based sterilization. With steam sterilization, instruments/implants are placed within a sterilization tray and loaded into a sterilization device, such as an autoclave. Using a combination of steam, time, temperature and pressure, the instruments/implants in the autoclave are sterilized.

In these embodiments, the enclosure of the present invention is capable of withstanding the high temperature and pressures of steam sterilization. Steam sterilization is typically achieved by exposing products to saturated steam at high temperatures (about 250° F. to about 275° F.). The Products are placed in a device called the autoclave and heated through pressurized steam to kill all microorganisms including spores. In particular, the embodiments may be designed to withstand temperatures of at least about 270° Fahrenheit for at least 60 consecutive minutes without compromising the design of the system. Accordingly, in the embodiments, the enclosure of the present invention will have a high heat deflection value, i.e., little deformation and dimensional change due to the extended heating for a high number of cycles.

Sterilization Container Features

With regard to the container, which may take the form of a tray, basket or other known vessel for holding instruments for sterilization, the surgical or dental instruments/implants may be held within the sterilization container in spaced relation to each other and within the walls of the container. The container may be formed with a width, length, height, and depth designed to accommodate different types of instruments and implants. In addition, the container may contain a variety of supports, clamping members, forms, mats, pressure skids, and other instrument retaining means. These structures may be shaped in a variety of configurations, such as horizontally, vertically, and compartmentalized. In some embodiments, the container may comprise various holes or slots used as locating tabs for small barriers or walls to allow for internal configurations, e.g., sub-trays within the main tray.

In some embodiments, the sterilization container may be constructed from metal, such as stainless steel, plastic or other materials suitable for medical device sterilization procedures. Metal sterilization containers may be used in the embodiments, for example, if it is desired for the tray to retain its shapes during high temperature steam sterilization. Alternatively, in some embodiments, the container may be constructed from a plastic, such as Radel® 5000. Plastic containers may be used in the embodiments when it is desired to provide a pliable structure that will not damage sensitive instruments or implants, as may occur when using metal containers.

Adaptive Enclosure—a Tray “Skin”

With regard to the outer sleeve or collapsible enclosure, in some embodiments, the enclosure is provided as a thermoplastic wrap or skin that is sized/shaped to conform to the sterilization container (hereinafter a “tray skin”). In other words, in some embodiments, the enclosure is a protective barrier and acts as a “skin” that can conform and adapt to the size and shape of the container. Of course, while described as a “tray skin” it is understood that such term does not limit the outer sleeve to use with trays only, as the outer sleeve can be used with all types of sterilization containers including baskets and other similar holding structures. Further, the container may or may not have a lid or cover.

In some embodiments, the tray skin design is tailored to fit the need of a surgical or dental medical facility, as well as other validated uses with similar requirements for sterile packaging. In particular, the tray skin serves as a flexible particulate and fluid barrier designed to maintain sterility, especially during and following a steam exposure cycle and/or other similarly validated sterile procedures. Accordingly, the tray skin allows surgical related medical facilities utilizing steam sterilization technology or similarly validated sterilization methods to be able to sterilize their instruments, implants, or other similar validated items in any sterile packaging, container, or similar enclosure they prefer.

The tray skin of the present invention reduces or eliminates drying and cool-down times and obviates the need for equalizing the tray. This can potentially save the medical facility anywhere from 1.5 to 3 hours in down-time, thereby providing more capacity for emergent or unplanned procedures. In addition, the tray skin eliminates “wet loads” because the only moisture that remains in the sterilization container is “sterile condensation” caused by condensation on surface of the metal components (i.e., outside moisture is not present within the interior of the tray skin) when the sterilizer is opened from 270° to room temperature due to reducing or eliminating the need for a dry and cool down cycle. (i.e., the entire inside and outside of the sterilization container is within the sterile field). The tray skin by design is a particulate and fluid barrier when in the closed position protecting anything inside of the tray skin from outside foreign contaminants under approved regulatory sterilization parameters. In certain embodiments, the tray skin is designed as a disposable, one-time use item that can be thrown away after use.

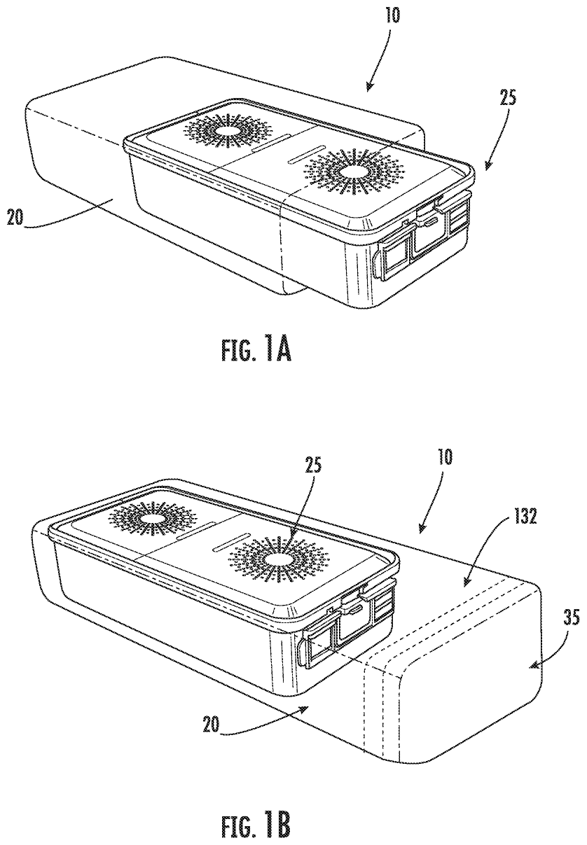

Referring now to FIGS. 1A and 1B, an outer sleeve or cover 10 according to one embodiment of the present invention includes a collapsible enclosure 20 for housing a representative sterilization container 25 within an interior 30 of enclosure, and a sealable opening 35 for moving the sterilization container into and out of interior 30. Collapsible enclosure 20 is flexible and designed to conform to multiple sizes to fit around a variety of different containers. In preferred embodiments, enclosure 20 has an open or enlarged size of about 10 inches to 20 inches in width and about 10 inches to 30 inches in length, although it should be recognized that enclosure is not limited to these sizes. In an exemplary embodiment, enclosure 20 resembles a lightweight square or rectangular shaped duffle bag or collapsible suitcase, although other configurations can be used in accordance with the present invention.

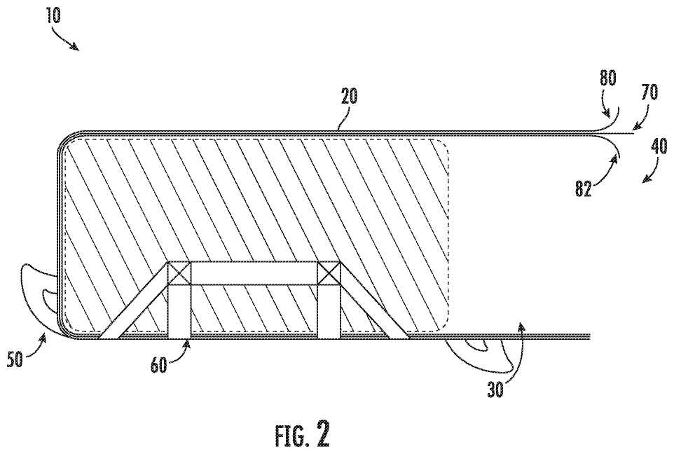

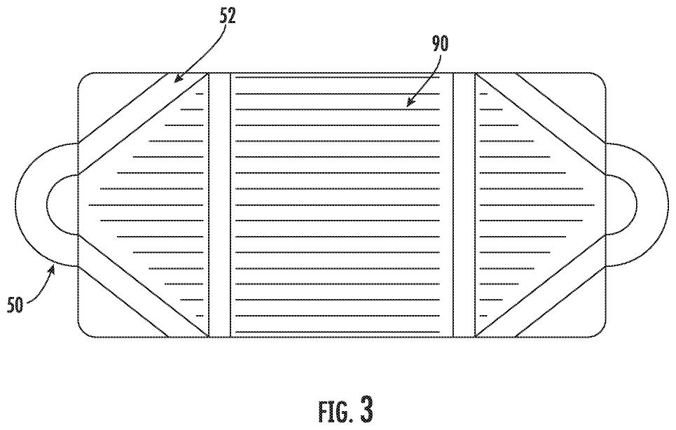

In certain embodiments (shown in FIGS. 2 and 3), enclosure 20 further includes one or more handles 50 and webbing supports 60 to compliment the tray structure, depending on their intended use. Handles 50 and weight supports 60 may be connected together and secured to enclosure 20. In some embodiments, these structures are made of a durable non-absorbent webbing material, such as a thermoplastic or firm elastomer, designed to provide support for the weight of items placed inside of the tray.

In an exemplary embodiment, handles 50 are circularly shaped like a narrow racetrack with two elongate portions 52 extending along the bottom and midway up the sides of enclosure 20 (see FIG. 2). Handles 50 and weight supports 60 may be thermally bonded to enclosure 20 and are preferably designed to withstand temperatures of at least 270 degrees Fahrenheit for a minimum of 60 consecutive minutes.

Of course, it should be understood that enclosure 20 may include features other than handles for carrying sleeve, such as a strap, sling, belt, cord, buckle, band, tie, wheels (i.e., for a rolling bag) or the like. Alternatively, enclosure 20 may include a single handle, or more than 2 handles. In addition, enclosure 20 may include other features, aside from webbing supports 60 to provide support for enclosure 20, such as additional protective layers at selected locations around enclosure 20.

Referring now to FIG. 2, enclosure 20 is a tray skin that comprises a sealable skin/outer sleeve having a multi-layer structure comprising one or more middle layers 70 and one or more protective layers 80, 82. In an exemplary embodiment, the one or more middle layers 70 of the tray skin are made from a non-woven polypropylene, non-absorbable hydrophobic spun lace anti-microbial material. Of course, it will be recognized that a variety of other suitable thermoplastic or thermoset polymers that meet the requirements of the present invention may be used for middle layer 70.

In certain embodiments, middle layer 70 may be designed with a hydrostatic head with specified and engineered PSI creating anti-wicking properties that form both a particulate/pathogen barrier and a liquid barrier. In addition, middle layer 70 still allows steam and other validated sterilization methods to pass through the material to sterilize its contents. Thus, middle layer 70 is permeable to certain gases, such as steam, while being substantially impermeable to liquids, pathogens and other particulates. Accordingly, middle layer 70 of the tray skin creates a barrier that protects the items in the enclosure from outside moisture, contaminates, and/or pathogens that could affect the sterility of the contents held within the tray. As noted above, middle layer 70 of enclosure 20 is designed to withstand a minimum of 270 degrees Fahrenheit for a minimum of 60 consecutive minutes.

Protective layers 80, 82 preferably comprise an outer layer 80 and an inner layer 82 configured to protect and substantially surround middle layer 70. Protective layers 80, 82 are made of a strong, breathable, hydrophobic material with a tensile strength designed to withstand the high temperature and pressure environments found in sterile processing applications, such as an autoclave. In an exemplary embodiment, protective layers 80, 82 comprise a canvas-like material made of cotton, linen, hemp or the like, although it will be appreciated that other suitable materials may be used, such as denim, vinyl or the like, In one embodiment, protective layers 80, 82 are spot-bonded to middle layer 70 with a medically safe bonding material. This bonding allows the layers to be secured to each other without affecting the integrity and functionality of its design. Protective layers 80, 82 are designed to withstand temperatures of at least 270 degrees Fahrenheit for at least 60 consecutive minutes. Protective layers 80, 82 preferably cover substantially the entire portion of each side of middle layer 70. However, it will be understood that other configurations are possible. For example, protective layers 80, 82 may cover only certain portions of middle layer 70, i.e., a sufficient amount of coverage to protect the middle layer 70.

As shown in FIG. 3, enclosure 20 may include pressure skids 90 to provide a structure to bear the weight of an instrument or implant. Accordingly, pressure skids 90 are designed with various shapes and sizes to absorb the pressure of heavy objects, and distribute the pressure away from the layers of the tray skin. In one embodiment, pressure skids 90 are made with a medically-safe, hardened polymer material, such as an elastomer/rubber, or similar approved material that penetrates and hardens through the tray skin and serves to redirect pressure.

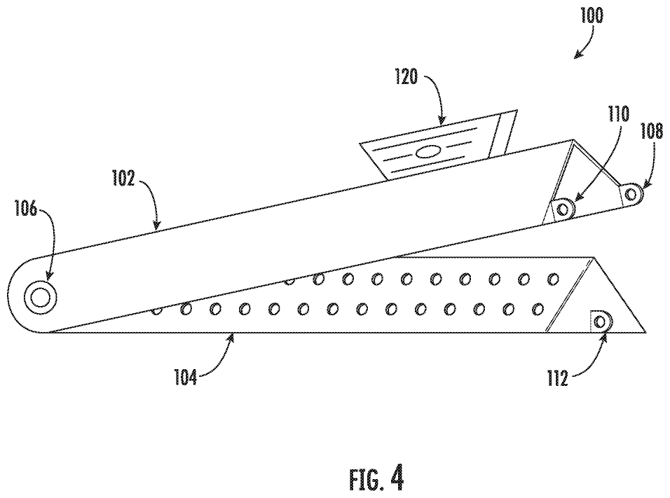

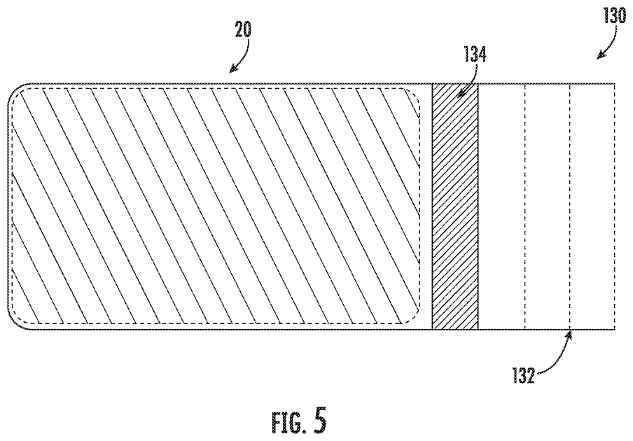

Referring now to FIGS. 4 and 5, enclosure 20 includes a sealable opening for allowing the sterilization container to be moved into and out of enclosure 20. In the representative embodiment, the sealable opening is a substantially rectangular-shaped fold seal on the side of enclosure 20, although other configurations may be used (e.g., triangular-shaped, oval, circular, etc.).

In one embodiment (shown in FIG. 4), enclosure 20 comprises a seal clip 100 made from a durable medically safe polymer or other suitable material. Seal clip 100 preferably comprises upper and lower clamps 102, 104 pivotally coupled to each other with a hinge 106. Upper clamp 102 includes a pair of outer locking barrels 108, 110 and lower clamp 104 includes a central locking barrel 112. Outer locking barrels 108, 110 can be secured to central locking barrel 112 to lock clamps 102, 104 into position and secure clip 100. A handle 120 may be formed on the upper or lower clamp to facilitate moving clamps 102, 104 into the closed position.

Clamps 102, 104 preferably have a length of about 10 to 30 inches, although the specific size will depend on the size of the sterilization containers housed within enclosure 100. In one embodiment, upper clamp 102 is generally triangular-shaped and provides a press-fit slide and secure click over lower clamp 104. As shown, there is an overlapping keyhole design on the secured end of enclosure 20 to secure and seal clip 100, which extends past enclosure 20 to provide for a pre-engineered tamper proof lock, thereby ensuring that the enclosure 20 has remained secure from the time it was sealed until the time the enclosed items are needed for use.

In another embodiment (shown in FIG. 5), enclosure 20 comprises an outer portion 130 that may be folded relative to the rest of enclosure 20. Outer portion 130 may include one or more pre-marked fold guides 132 for ease of use. Enclosure 20 further comprises a self-adhesive strip 134 designed to adhere to outer layer 80 of enclosure 130. Self-adhesive strip 134 may comprise any suitable material, such as epoxy, acrylic, rubber, silicone, polyurethane or the like. In use, the operator folds outer portion 130 over enclosure 20 and seals enclosure 20 by firmly pressing along adhesive strip 134 to provide even coverage to seal.

In certain embodiments, enclosure 20 further includes a sensor (not shown) for detecting pathogens, liquids or other particulate matter within the interior of enclosure. Suitable sensors for use with the present invention may include PCT and microarray based sensors, optical sensors (e.g., biolouminescence and fluorescence), piezoelectric, potentiometric, amperometric, conductometric, nanosensors or the like. Enclosure 20 further includes an indicator, such as a display on the outer surface of enclosure 20 (not shown), coupled to the sensor and configured to indicator the presence of pathogens, liquids or other particulars detected by the sensor. The indicator may be any suitable chemical indicator validated for sterilization procedures that undergoes a physical or chemical change visible to the human eye after exposure to certain parameters. The indicator and sensor may be part of the same device, or separate from each other. The sensor is preferably designed to detect pathogens after clip 100 has been sealed and moved into a closed position to ensure that enclosure 20 has remained secure from the time it was sealed until the time the enclosed instruments or other items are needed for use.

In use, enclosure 20 is place around a sterilization container, such as the container 25 shown in FIG. 1. The container 25 can be advanced into enclosure 20 until it is completely enclosed within the interior 30 of cover 10. Sealable opening 35 extends beyond container 25, as shown in FIG. 1. Sealable opening 35 includes one or more fold lines or guides 132 to facilitate the operator's closing of sealable opening 35 over the end of container 25 such that container 25 is completed enclosed within cover 10. Sealable opening 35 is then closed and sealed, either with seal clip 100, self-adhesive strip 134 or other suitable means. Once container 25 is secured within cover 10, a tamper-proof sterilization chemical lock (such as the chemical indicator described above) is engaged to ensure that the interior of cover remains sterile until it is ready for use.

While the invention has been described in detail herein in accordance with certain preferred embodiments thereof, many modifications and changes therein may be effected by those skilled in the art. Accordingly, the foregoing disclosure should not be construed to be limited thereby but should be construed to include such aforementioned obvious variations and be limited only by the spirit and scope of the following claims.

1. A sterilization cover comprising:
a collapsible enclosure configured to conform around an outer surface of a sterilization container, the enclosure comprising an inner layer configured to withstand a sterilization procedure and a protective layer bonded to the inner layer and configured to protect the inner layer and to withstand the sterilization procedure; and
a sealable opening for receiving the sterilization container within the enclosure.

2. The cover of claim 1, wherein the sterilization procedure includes steam sterilization.

3. The cover of claim 1 wherein the sterilization procedure includes hydrogen peroxide, gas plasma, ozone or ethylene oxide.

4. The cover of claim 1, wherein the sealable opening is movable between an open position, wherein the opening is configured to receive the sterilization container within the collapsible enclosure, and a closed position, wherein the opening is substantially impermeable to liquids and pathogens.

5. The cover of claim 1, wherein the inner layer and the protective layer are configured to withstand a temperature of at least 270 degrees Fahrenheit for at least 60 minutes.

6. The cover of claim 1, wherein the inner layer and the protective layer each comprise a material that is permeable to gases and substantially impermeable to liquids and pathogens.

7. The cover of claim 1, wherein the protective layer comprises a first layer bonded to one side of the inner layer and a second layer bonded to an opposite side of the protective layer.

8. The cover of claim 1, wherein the inner layer comprises a substantially non-absorbable, anti-microbial material.

9. The cover of claim 1, wherein the inner layer comprises a non-woven polypropylene, hydrophobic material.

10. The cover of claim 1, wherein the outer layer comprises a material with substantially high tensile strength.

11. The cover of claim 3, wherein the sealable opening comprises a self-adhesive portion for bonding to the outer layer.

12. The cover of claim 3, wherein the sealable opening comprises a clamp.

13. The cover of claim 11, wherein the clamp further comprises a lock for locking the clamp in the closed position.

14. The cover of claim 3, further comprising a sensor configured to detect a presence of a pathogen within an interior of the enclosure when the sealable opening is in the closed position.

15. The cover of claim 13, further comprising an indicator coupled to the sensor and configured to indicate the presence of the pathogen within the interior.

16. The cover of claim 1, further comprising a handle coupled to the outer layer.

17. The cover of claim 1, further comprising a pressure skid surface on at least one portion of the enclosure.

18. An outer sleeve for use with a sterilization container, comprising:
a flexible inner layer configured to conform to an outer surface of the sterilization container and comprising a material that is substantially impermeable to liquids and pathogens and permeable to gases;
an outer layer bonded to the inner layer and comprising a material configured to protect the inner layer; and
wherein the inner and outer layers define an interior and comprise a sealable opening for receiving the sterilization container within the interior.

19. The outer sleeve of claim 17 wherein the protective layer comprises a material with a high tensile strength, the material being permeable to gases and substantially impermeable to liquids and pathogens.

20. The outer sleeve of claim 17 wherein the sealable opening is movable between an open position, wherein the opening is configured to receive the sterilization container within an interior of the sleeve, and a closed position, wherein the sealable opening is substantially impermeable to liquid and pathogens.

21-29. (canceled)
